(19) United States
(12) Patent Application Publication
(10) Pub. No.: US 2007/0077593 A1
(43) Pub. Date: Apr. 5, 2007

(54) Substrate as a ligate carrier

(75) Inventor: Gerhard Hartwich, Munich (DE)

Correspondence Address: CROCKETT & CROCKETT, 24012 CALLE DE LA PLATA, SUITE 400, LAGUNA HILLS, CA, 92653, US

(73) Assignee: FRIZ Biochen Gesellschaft fur Bioanalytik mbH, MUNCHEN (DE)

(21) Appl. No.: 10/578,661

(22) PCT Filed: May 7, 2004
(86) PCT No.: PCT/EP04/04906

§ 371 Date: May 8, 2006

(30) Foreign Application Priority Data
May 7, 2003 (DE) 103 20 312.5

(51) Int. Cl. G01N 33/53; C12M 1/34

(52) U.S. Cl. 435/7.1; 435/287.2

(57) ABSTRACT

Described is a substrate for use as a ligate carrier in a method for detecting ligate-ligand association events, having test sites 24 disposed on the substrate and having ligates 26 bound to the surface of the test sites 24, at least two types of test sites 24 being provided, the different types of test sites each being loaded with different types of ligates 26, the different types of ligates 26 detecting the respective complementary types of ligands, the ligands being present in an analyte solution in different concentration ranges in each case, and the test sites 24 exhibiting a characteristic loading parameter that permits detection of the ligands in their respective concentration range.

### FIELD OF THE INVENTION

The invention relates to a substrate for use as ligate carrier.

### BACKGROUND OF THE INVENTION

In the field of biosciences, medical devices and sensor technology, many sensors and methods have been developed for genomics and proteomics research, especially in recent years. To understand organisms, it is essential to analyze their genes or their protein set. Humans, for example, have some 30,000 to 50,000 genes and about 500,000 different proteins. To be able to detect this enormous information content, sensors having a high degree of parallelization and intelligent analysis algorithms are needed. A key limitation with regard to the quality of a sensor is the so-called “dynamic range” of the sensor.

The term “dynamic range” of a sensor is understood to be the range in which the sensor reacts reproducibly and specifically to changes in the concentration of a certain analyte. The “dynamic range” of a sensor is normally about a factor of 10 to 100 in the analyte concentration, and is limited for smaller concentrations by the sensitivity of the detection method. For high concentrations, the sensor reaches saturation above a certain range, such that a further increase in the analyte concentration triggers no signal change.

In the field of gene expression analysis of organisms or identification of foreign germs, such as viruses or bacteria in organisms, as done, for example, in medical examinations, the problem often arises of having to quantitatively analyze many different analytes in parallel. However, the concentrations of these analytes can fluctuate by many orders of magnitude. The analytes are already present in very different concentrations in the non-pathogenic state. The development of a pathogenic different analytes in parallel. However, the concentrations of these analytes can fluctuate by many orders of magnitude. The analytes are already present in very different concentrations in the non-pathogenic state. The development of a pathogenic effect normally begins only when an analyte-dependent limit is exceeded, which can be many times higher than the tolerable base analyte concentration. Such extremely scattered changes in the analyte concentrations cannot be detected in parallel by the sensors known from the background art. Rather, multiple sensors are used, each of which detects only changes in the concentration of one group of analyte molecules that are present in a similar initial concentration.

### DESCRIPTION OF THE INVENTION

This is where the present invention begins. The object of the present invention, as characterized in the claims, is to provide a sensor that facilitates parallel detection of the concentration fluctuations of components of an analyte fluid, these components being able to be present in the test substance in concentrations that differ by orders of magnitude.

According to the present invention, this object is solved by the substrate according to claim 1 and the use of the substrate according to claim 24. Further advantageous details, aspects and embodiments of the present invention are evident from the dependent claims, the description, the drawings and the examples.

The following abbreviations and terms will be used in the context of the present invention:

| | |
|---|---|
| ACV | alternating current voltammetry |
| dynamic range | the range of a sensor in which it reacts reproducibly and specifically |
| | to changes in the concentration of a certain analyte |
| characteristic loading | Parameter of the active regions of the sensor surface, such |
| parameter | as the geometric surface area of the test sites or their |
| | loading density with ligates. The characteristic loading |
| | parameter defines the number of respective ligates on the |
| | sensor surface and thus, via the association constant, also |
| | the number of association events for a given analyte |
| | concentration. |
| FcAc | ferrocene acetic acid |
| fluorophore | A chemical compound (chemical substance) that is capable |
| | of emitting, upon excitation with light, a longer-wave (red- |
| | shifted) fluorescent light. Fluorophores (fluorescent dyes) |
| | can absorb light in a wavelength range from the ultraviolet |
| | (UV) to the visible (VIS) to the infrared (IR) range. The |
| | absorption and emission maxima are typically shifted against |
| | each other by 15 to 40 nm (Stokes shift). |
| laser ablation | partial or complete removal of organic or inorganic |
| | passivation layers, as well as the removal of impurities on a |
| | substrate by irradiation with laser light |
| ligand | Refers to molecules that are specifically bound by a ligate; |
| | examples of ligands within the meaning of the present |
| | invention are substrates, cofactors and coenzymes of a |
| | protein (enzyme), antibodies (as the ligand of an antigen), |
| | antigens (as the ligand of an antibody), receptors (as the |
| | ligand of a hormone), hormones (as the ligand of a receptor) |
| | and nucleic acid oligomers (as the ligand of the |
| | complementary nucleic acid oligomers). |
| ligate | Refers to a (macro-)molecule on which are located specific |
| | recognition and binding sites for the formation of a complex |
| | with a ligand. Examples of ligates within the meaning of the |
| | present invention are substrates, cofactors and coenzymes |
| | of a protein (enzyme), antibodies (as the ligate of an |
| | antigen), antigens (as the ligate of an antibody), receptors |
| | (as the ligate of a hormone), hormones (as the ligate of a |
| | receptor) and nucleic acid oligomers (as the ligate of the |
| | complementary nucleic acid oligomers). |
| μCP | microcontact printing |
| osmium complex | [Os(bipy)₂ Cl imidazoleacrylic acid] |
| SDS | sodium dodecyl sulfate |
| probe | biomolecules applied to the sensor surface that can |
| | specifically bind one or more molecules from the test |
| | substance (targets) |
| spacer | Any molecular link between two molecules or between a |
| | surface atom, surface molecule or surface molecule group |
| | and another molecule. It is normally an alkyl, alkenyl, |
| | alkynyl, heteroalkyl, heteroalkenyl or heteroalkynyl chain. |
| | Preferred spacers are those having a chain length of 1-20, |
| | especially a chain length of 1-14, the chain length |
| | representing the shortest continuous link between the |
| | structures to be linked. |
| spot or | Spatially limited regions on the sensor surface that each |
| test site | carry one or more types of probe molecules that can each |
| | specifically bind one or more molecules of a test substance. |
| | It is possible to optimize the size of these regions or their |
| | surface loading with probes to the order of magnitude of the |
| | target concentration. |
| target | molecules in the test substance that can specifically bind to |
| | one or more biomolecules on the sensor surface (probes) |
| (n × HS-spacer)-oligo | A nucleic acid oligomer to which n thiol functions are each |
| | attached via a spacer, each spacer being able to exhibit a |
| | different chain length (the shortest continuous link between |
| | the thiol function and the nucleic acid oligomer), especially |
| | any chain length between 1 and 14 each. These spacers, in |
| | turn, can be bound to different reactive groups that are |
| | naturally present on the nucleic acid oligomer or that have |
| | been affixed thereto by modification. Here, “n” is any integer, |
| | especially a number between 1 and 20. |
| (n × R-S-S-spacer)- | A nucleic acid oligomer to which n disulfide functions are |
| oligo | each attached via a spacer, the disulfide function being |
| | saturated by any residue R. Each spacer for attaching the |
| | disulfide function to the nucleic acid oligomer can exhibit a |
| | different chain length (shortest continuous link between the |
| | disulfide function and the nucleic acid oligomer), especially |
| | any chain length between 1 and 14. These spacers, in turn, |
| | can be bound to different reactive groups that are naturally |
| | present on the nucleic acid oligomer or that have been |
| | affixed thereto by modification. The variable “n” is any |
| | integer, especially a number between 1 and 20. |
| oligo-spacer-S-S- | Two identical or different nucleic acid oligomers that are |
| spacer-oligo | linked with each other via a disulfide bridge, the disulfide |
| | bridge being attached to the nucleic acid oligomers via any |
| | two spacers, and the two spacers being able to have |
| | differing chain lengths (the shortest continuous link between |
| | the disulfide bridge and the respective nucleic acid |
| | oligomer), especially any chain length between 1 and 14 |
| | each. These spacers, in turn, can be bound to different |
| | reactive groups that are naturally present on the nucleic acid |
| | oligomer or that have been affixed thereto by modification. |
| DNA | deoxyribonucleic acid |
| RNA | ribonucleic acid |
| PNA | peptide nucleic acid (Synthetic DNA or RNA in which the |
| | sugar-phosphate moiety is replaced by an amino acid. If the |
| | sugar-phosphate moiety is replaced by the |
| | —NH—(CH₂)₂—N(COCH₂-base)-CH₂CO— moiety, PNA will |
| | hybridize with DNA.) |
| A | adenine |
| G | guanine |
| C | cytosine |
| T | thymine |
| base | A, G, T or C |
| bp | base pair |
| nucleic acid | At least two covalently linked nucleotides or at least two |
| | covalently linked pyrimidine (e.g. cytosine, thymine or uracil) |
| | or purine bases (e.g. adenine or guanine). The term nucleic |
| | acid refers to any “backbone” of the covalently linked |
| | pyrimidine or purine bases, such as the sugar-phosphate |
| | backbone of DNA, cDNA or RNA, a peptide backbone of |
| | PNA, or analogous structures (e.g. a phosphoramide, |
| | thiophosphate or dithiophosphate backbone). An essential |
| | feature of a nucleic acid within the meaning of the present |
| | invention is the sequence-specific binding of naturally |
| | occurring cDNA or RNA. |
| nucleic acid oligomer | A nucleic acid of a base length that is not further specified |
| | (e.g. nucleic acid octamer: a nucleic acid having any |
| | backbone in which 8 pyrimidine or purine bases are |
| | covalently bound to one another). |
| oligomer | equivalent to nucleic acid oligomer |
| oligonucleotide | equivalent to oligomer or nucleic acid oligomer, in other |
| | words e.g. a DNA, PNA or RNA fragment of a base length |
| | that is not further specified |
| oligo | abbreviation for oligonucleotide |
| ss | single strand |
| K | association constant |
| [S] | actual loading density of the probe molecules on the surface |
| | after binding of the ligands to the ligates |
| [ST] | loading density of the complexes composed of target and |
| | probe molecule on the surface |
| S₀ | total loading density of the probe molecules on the surface |
| T | target concentration |

The present invention relates to a substrate for use as a ligate carrier in a method for detecting ligate-ligand association events. Disposed on the substrate are test sites that exhibit ligates that are bound to the surface. At least two types of test sites are provided, the individual test sites being loaded with different types of ligates. These different types of ligates detect the respective complementary types of ligands, which are present in an analyte solution in different concentration ranges. The test sites exhibit a characteristic loading parameter, such that detection of the ligands is facilitated in the concentration range in which the respective ligand is present in the analyte solution.

A sensor having a given number of specific coupling sites reaches a saturation value above a certain analyte concentration in the test substance, such that a further increase in the concentration can no longer be detected. This can be described, in theory, with first-order binding kinetics for the binding of a probe S on the sensor surface and a target T in the test substance to a surface complex ST having an association constant K:
K=[ST]/[S].[T] (1)

For the surface loading densities, [S]=S₀−[ST], where S₀ is the total loading density of the probes and [S] is the loading density of free probes in thermodynamic equilibrium. Converting the above equation, an expression [ST]/S₀ is obtained for the relative proportion of surface complexes:
[ST]/S₀=K.[T] /(1+K.[T])=1−[S]/S₀ (2)

This expression as a function of the surface loading density S₀ normalized to the association constant K is illustrated in FIG. 1 for four different target concentrations [T] and corresponds to the known Langmuir binding isotherms. However, in the case of binding events that are no longer independent of one another and whose binding energies are subject to a distribution, the Langmuir model no longer applies. Heterogeneous adsorption isotherms develop that are described, for example, by the Sips model:
[ST]/S₀=(K.[T])^{a}/(1+(K.[T])^{a}) (3)
where a is a parameter (a≦1) representing a pseudo-Gaussian distribution of binding energies. For a =1, the above formula changes back to the Langmuir isotherm.

The illustration of the Langmuir model (FIG. 1) clearly shows that, for a given analyte concentration, the proportion of binding events does not increase any further above a probe count less than a certain limit. This range is referred to as “ambient analyte conditions” (U.S. Pat. No. 5,807,755). The number of probes on the sensor leads to no marked depletion (less than 10%) of the targets in the test substance. Under “ambient analyte conditions,” an increase in the analyte concentration leads only to a parallel shift of the plateau to higher proportions of binding events. After an increase by 2 to 3 orders of magnitude, the saturation of the sensor is reached.

Thus, for a given association constant and sensor probe count, if an analyte concentration is present that leads to a relative loading (cf. [TS]/S₀ in FIG. 1) of close to 1, then further increases in the concentration of this species in the test substance are no longer detectable. However, if the sensor probe count is increased for a test substance target type that is present in too high a concentration, or if these probes are applied to larger electrodes at the same loading density, it is possible to reduce the relative loading of this reaction and thus optimize the sensitivity.

The present invention provides a sensor having, with a view to analyzing analyte fluids that contain analytes in very different concentrations, an optimized “dynamic range”.

The present invention is based on the idea that, by optimizing the “dynamic range” of a sensor, changes in the concentrations of components of a liquid test substance can also be detected in parallel when the initial concentrations of these components are scattered over many orders of magnitude.

The substrate of the present invention is preferably used as a biomolecule carrier in a method for electrochemically or fluorescent spectroscopically detecting components of an electrolyte solution. The substrate according to the present invention can also be used in an electrochemical or fluorescent spectroscopic method for detecting biomolecules.

The present invention describes a sensor having spatially limited regions of different probe molecules (spots) that can each specifically bind one or more target molecules from a test substance. The spots of the present invention are so optimized in size and/or probe surface loading (characteristic loading parameter of the substrate) to the corresponding targets′ concentration ranges to be detected that the proportion of binding events of all spots for, for example, the non-pathogenic state is approximately identical, independent of the actual concentration of their targets. In this way, it is possible to normalize the specific “dynamic range” of a sensor to this “initial state”. The advantage of this method lies in the adapted sensitivity of all spots with a view to the corresponding targets′ concentration changes to be detected, independent of their initial concentration. The sensor is thus optimized to the “tolerable” concentration range between the “permitted,” non-pathogenic value and the “critical,” pathogenic value of each analyte.

Thus, to optimize the sensor, the “permitted,” non-pathogenic concentrations of the analytes in the test fluids at the start of an experiment series should be known. Especially in the field of gene expression analysis or the identification of germs, the composition of the analyte pool of a healthy organism is normally sufficiently known, such that, with the present method, based on the changes of individual analytes, indications for a disease (exceeding the “critical” concentration value of an analyte) can be delivered.

For the quantitative readout of the different spots of the sensor with regard to possible binding events, within the scope of the present invention, all suitable measuring methods may be used, depending on the substrates used and the respective biomolecules.

Using the substrates according to the present invention, preferably different types of ligands are detected that are present in the analyte solution in concentration ranges whose mean values differ by at least a factor of 10. The mean value cm of a concentration range is understood to be the value cₘ=((cₘₐₓ−cₘᵢₙ)/2)+Cₘᵢₙ, where cₘₐₓ indicates the maximum concentration and cₘᵢₙ the minimum concentration. Preferably, different types of ligands are detected, the mean values of the concentration ranges in which they are present in the analyte solution differing by at least a factor of 100, especially preferably differing by at least a factor of 1,000, very particularly preferably differing by at least a factor of 10,000.

The present invention also comprises the use of the substrates in methods for detecting ligate-ligand association events.

The substrates can especially be used in fluorescent spectroscopic and electrochemical detection methods. Chronoamperometry (CA), chronocoulometry (CC), linear sweep voltammetry (LSV), cyclic voltammetry (CSV), alternating current voltammetry (ACV), voltammetry techniques with different pulse shapes, especially square wave voltammetry (SWV), differential pulse voltammetry (DPV) or normal pulse voltammetry (NPV), AC impedance spectroscopy, chronopotentiometry and cyclic chronopotentiometry can be used as electrochemical detection methods.

Sensor Substrate Having Active Areas of Different Sizes

According to a particularly preferred embodiment of the present invention, the characteristic loading parameter of the substrate is the size of the surface area of the individual test sites. Preferably, the surface area of the test sites differs by at least a factor of 10, particularly preferably by at least a factor of 100, especially preferably by at least a factor of 1,000 and very particularly preferably by at least a factor of 10,000.

Preferred are substrates that exhibit test site surface areas between 1 μm² and 1 mm². Particularly preferred are substrates that exhibit test site areas between 10 μm² and 100,000 μm².

Within the scope of the present invention, all solids having a freely accessible surface that can be functionalized with biomolecules and wetted with a liquid test substance are suitable as sensor substrates. Plastics as well as metals, semiconductors, glasses, composites and porous materials can be used as solid substrates. The term “surface” is independent of the spatial dimensions of the surface.

The surface of the sensor must be subdividable into spatially separate regions. This is realizable by structuring the solid substrate into active and inactive regions, or by partially functionalizing its homogeneous surface.

The structuring of the solid substrate into active and inactive regions is achievable, for example, through lithography, vacuum deposition, electrochemical deposition, doping or laser treatment. On homogeneous substrates, the structuring can be realized by applying and structuring passivation layers. According to the present invention, any material that forms a complete layer on a surface and thus separates the substrate surface from the surroundings is suitable as a passivation layer. The material can later be removed in its entire thickness without residue at the desired sites, for example by laser ablation.

Spatially separate regions of different functionalization can also be produced without structuring the substrates. By way of example, reference is made here to microcontact printing (μCP), which was first introduced by Whitesides 1994 (A. Kumar, G.M. Whitesides, Science, 1994, 263, 60.) In this method, a micropatterned stamp is wetted with a fluid, thereafter brought into direct contact with the substrate to be processed, and in this way, a lateral chemical pattern is stamped on the surface.

In a preferred embodiment of the present invention, electrically conductive materials such as platinum, palladium, gold, cadmium, mercury, nickel, zinc, carbon, silver, copper, iron, lead, aluminum, manganese, any doped or undoped semiconductors and binary or ternary compounds are used as surfaces of the sensor substrates. To realize spatially separate active test sites or spots on the sensor, homogeneous electrically conductive surfaces can be structured, or conductive materials can be applied in any thickness to spatially separate regions of a non-conductive substrate, such as glass or plastic.

According to a particularly preferred embodiment of the present invention, as sensor substrates, insulating support plates are used that are expediently one-sided rigid support plates, double-sided rigid support plates or rigid multilayer support plates. Alternatively, the insulating support plate can be a one-sided or double-sided flexible support plate, especially made of a polyimide film or a rigid-flexible support plate. It is advantageously composed of a base material that is selected from the group BT (bismaleimide triazine resin with silica glass), CE (cyanate ester with silica glass), CEM1 (hard paper core with FR4 outer layers), CEM3 (fiberglass mat core with FR4 outer layers), FR2 (phenolic resin paper), FR3 (hard paper), FR4 (epoxide woven glass fabric), FR5 (epoxide woven glass fabric with cross-linked resin system), PD (polyimide resin with aramide reinforcement), PTFE (polytetrafluoroethylene with glass or ceramic), CHn (highly cross-linked hydrocarbons with ceramic) and glass.

These support plates exhibit a certain number of conductor paths having a gold surface that are coated with a solder mask layer as passivation. Located at one end of the conductor paths are contacts for electrochemical analyses, and at the other end, free gold sites are burnt into the paint with laser ablation for the later functionalization. With the aid of laser ablation, spots of any size and geometry can be scribed in the paint, the only limit being the width of the conductor paths. According to the present invention, the laser ablation not only removes the coat of paint at the desired sites, but also, through the brief melting of the gold surface, ensures the reduction of the surface roughness and the closing of pores. Additionally, by melting the substrates, a few gold layers are ablated from the surface, and impurities thus removed.

The conductor path substrates just described are suitable both for electrochemical measuring methods and for fluorescent spectroscopy.

Functionalizing the Active Areas with Ligates

The active regions of the sensor are functionalized with ligates, which function as probes for the ligands present in the test substance. Within the scope of the present invention, all types of ligates are suitable for analyzing analyte fluids for the presence of their specific ligands. The term ligate refers to molecules that specifically interact with a ligand to form a complex. Examples of ligates within the meaning of the present text are substrates, cofactors or coenzymes, as complex binding partners of a protein (enzyme), antibodies (as complex binding partners of an antigen), antigens (as complex binding partners of an antibody), receptors (as complex binding partners of a hormone), hormones (as complex binding partners of a receptor), nucleic acid oligomers (as complex binding partners of the complementary nucleic acid oligomer) and metal complexes.

The background art provides a number of options for coupling the biomolecules to the sensor surface. Examples of this are: (i) thiol (HS) or disulfide (S—S) groups that couple to surfaces made of Au, Ag, Cd, Hg and Cu, (ii) amines that adsorb to platinum, silicon or carbon surfaces by chemisorption or physisorption, (iii) silanes that enter into a covalent bond with oxidic surfaces and (iv) epoxy cement that binds to all conductive surfaces (Heller et al., Sensors and Actuators, 1993, 180, 13-14; Pishko et al., Anal. Chem., 1991, 63, 2268; Gregg and Heller, J. Phys. Chem., 1991, 95, 5970-5975).

For immobilizing the probes, methods are preferred in which the number of probes on the sensor surface scales linearly with the surface area, so for example, applying probe monolayers under conditions that facilitate a loading smaller than the densest packing. However, within the scope of the present invention, “volume methods” for immobilizing the probes, for example via functionalized polymers, are also conceivable, as long as the number of probes continues to scale with the surface area.

The free substrate sites are preferably wetted with modified nucleic acid oligomers in aqueous solution. The nucleic acid oligomer that is to be applied to the free surface is modified with one or more reactive groups via a covalently attached spacer of any composition and chain length, these reactive groups preferably being located near one end of the nucleic acid oligomer. The reactive groups are preferably groups that can react directly with the unmodified surface. Examples of this are: (i) thiol- (HS—) or disulfide- (S—S—) derivatized nucleic acid oligomers having the general formula (n x HS-spacer)-oligo, (n×R—S—S—spacer)-oligo or oligo-spacer—S—S—spacer-oligo that react with a gold surface to form gold-sulfur bonds, (ii) nucleic acid oligomers having amines that adsorb to platinum or silicon surfaces through chemisorption or physisorption and (iii) nucleic acid oligomers having silanes that enter into a covalent bond with oxidic surfaces. Normally, loadings smaller than the densest packing are realized with these types of attachment of nucleic acid oligomers, such that sufficient space is available on the surface for a later hybridization.

At the other end of the nucleic acid oligomer, if needed, the molecule can additionally be modified with an electrochemical label via a further spacer of any composition and chain length if the functionalization of the free substrate sites and the later hybridization are to be analyzed with the aid of electrochemical methods. Electrochemical methods can also be used to analyze the hybridization events without modifying the probe oligonucleotides with a redox label if, alternatively, the target molecules are provided with a redox label. A further electrochemical detection variant is a displacement assay, in which short-chain signal oligomers bound to the unlabeled probe oligomers and having a redox label are displaced by unlabeled target oligomers of the complementary sequence.

As redox labels of the ligates or ligands transition metal complexes, especially those of copper, iron, ruthenium, osmium or titan with ligands such as pyridine, 4,7-dimethylphenanthroline, 9,10-phenanthrenequinonediimine, porphyrins and substituted porphyrin derivatives may be used. In addition, it is possible to employ riboflavin; quinones such as pyrroloquinoline quinone, ubiquinone, anthraquinone, naphthoquinone or menaquinone, or derivatives thereof; metallocenes and metallocene derivatives, such as ferrocenes and ferrocene derivatives, cobaltocenes and cobaltocene derivatives; porphyrins; methylene blue; daunomycin; dopamine derivatives; hydroquinone derivatives (para- or ortho-dihydroxybenzene derivatives, para- or ortho-dihydroxyanthraquinone derivatives, para- or ortho-dihydroxynaphthoquinone derivatives); and similar compounds.

Alternatively to the redox label, ligates or ligands can receive a fluorophore as a second functionalization, via a further spacer of any composition and chain length, if the functionalization of the free substrate sites and the later hybridization are to be analyzed with the aid of optical methods. Analogously to electrochemical analysis, fluorescent spectroscopy can also be carried out with a fluorophore at the target molecules and unlabeled probes.

For this, commercially available fluorescent dyes such as Texas Red®, rhodamine dyes, Cy3™, Cy5™, fluorescein, etc. (cf. Fluka, Amersham and Molecular Probes catalog) can be used.

Two techniques in particular are suitable for functionalizing the exposed substrate sites. In the spotting method, small volumes are specifically applied to the spots on the substrate with a commercially available spotter, each spot being able to be functionalized with different molecules. Alternatively, all exposed spots can be functionalized with identical probe molecules in that, for example, the substrate is immersed in the probe fluid or the entire substrate is wetted.

Varying the Surface Concentration of the Ligates

According to a further embodiment of the present invention, the number of probes on the sensor surface can also be adjusted without varying the active spot size. In this way, different amounts of probe molecules can also be realized on spots of identical size with only one sensor design.

Thus, substrates whose characteristic loading parameter is the loading density of the test sites with ligates constitute a particularly preferred embodiment of the present invention.

Particularly preferably, the loading densities of the test sites with ligates differ by at least a factor of 10, especially preferably by at least a factor of 100 and very particularly preferably by at least a factor of 1,000.

Different variants are suitable for controlling the surface loading. The loading can be adjusted, for example, via the incubation time, the number of coupling groups per molecule, the molarity of the loading buffer or via the concentration of the molecules in the incubation solution.

According to a preferred embodiment of the present invention, the surface loading is adjusted via a coadsorbate. For this, either a suitable coadsorbate is added in a certain concentration to the incubation solution of the probe molecules and brought into contact with the sensor surface, or the coadsorbate is applied with the probes in a second loading step following functionalization. The coadsorbate preferably exhibits the same coupling group as the probe molecule, and thus occupies a portion of the active surface and ensures a reduced surface loading of the probe. The surface loading can thus be adjusted via the concentration of the coadsorbate in the respective incubation solution.

For the above-described nucleic acid oligomers having thiol coupling groups, short-chain thiols having the general structure SH—(CH₂)ₙ—X, where X can be any head group, are particularly preferably suitable.

### BRIEF DESCRIPTION OF THE DRAWINGS

The invention will be explained in greater detail below by reference to exemplary embodiments in association with the drawings. Shown are:

FIG. 1 Theoretical curves of the relative proportion of binding events ([TS]/S₀) for different target concentrations. The concentration of the probes on the sensor surface is normalized to the association constant of the bond.

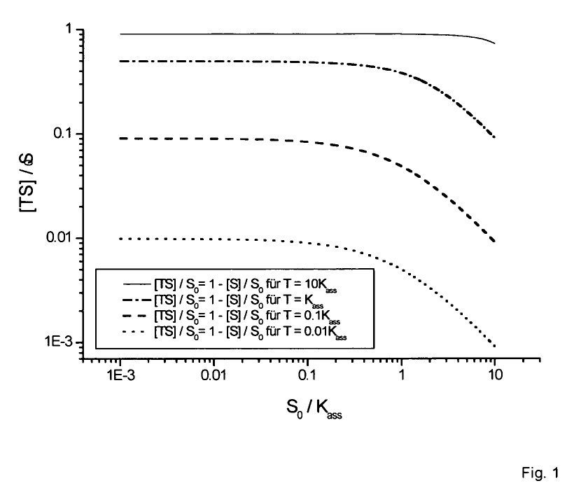

FIG. 2 Schematic image of a section of the sensor substrates based on printed circuit board technology. a) Top view on the conductor path substrate having free substrate sites of different active surface areas and geometries. b) Cross-section through a substrate having 3 identical electrode spots.

FIG. 3 a) Schematic image of a hybridization experiment with 2 redox labels. b) ACV curves (U_{ac}=10 mV, f=5 Hz) of a typical experiment on a working electrode with d=10 μm before and after hybridization. The left peak at about U=220 mV shows the osmium of the probe, the right peak at about U=360 mV shows the ferrocene of the target. The potentials are indicated against a Ag/AgCl reference electrode.

### MANNER OF EXECUTING THE INVENTION

An exemplary procedure for analyzing a test fluid having nucleic acid oligomers with the aid of a sensor based on printed circuit board technology is described in the following examples.

The gold sites of the substrate exposed by laser ablation are functionalized with doubly modified nucleic acid oligomers that have, at the one end, a thiol group for binding to the gold surface, and at the other end, an electrochemical label (e.g. osmium complexes). The desired number of probes on the sensor surface is adjusted either via the electrode size or by using a short-chain thiol of a certain concentration as a coadsorbate. The nucleic acid oligomers in the test fluid likewise have an electrochemical label (e.g. ferrocene derivatives), such that both the loading with the nucleic acid oligomers and the hybridization efficiency can be determined with electrochemical methods.

A preferred measuring method for analyzing the loading and the hybridization efficiency is AC (alternating current) voltammetry. According to O'Connor et al. (J. Electroanal. Chem., 466, 1999, 197-202) it is possible to calculate the number of labels involved from the ACV current at the redox potential of the label. The experiments are thus quantitatively analyzable.

For the person of skill in the art, the methods described in the following examples are easily transferable to the coating of other sensor surfaces with other biomolecules and to other detection methods.

### EXAMPLE 1

Printed Circuit Board Substrates

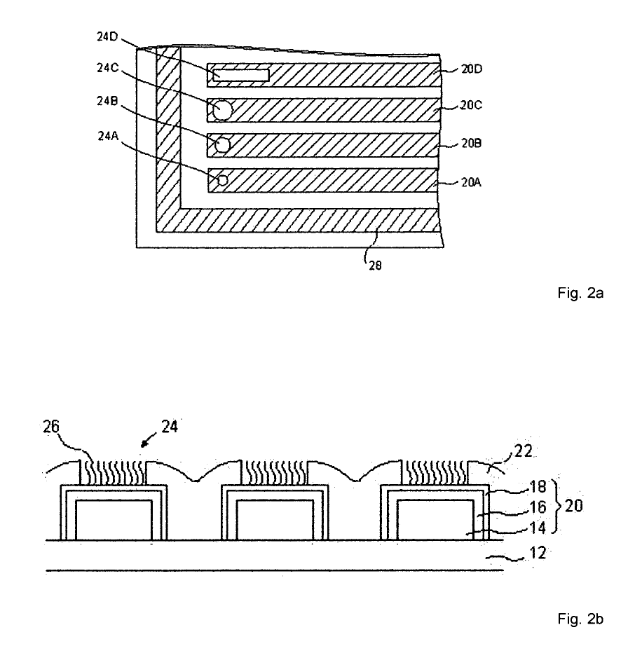

On a support plate made of epoxide woven glass fabric FR4 is applied a conductor pattern composed of fifty parallel conductor paths. FIG. 2a shows a section of this conductor path pattern. The section shows 4 of the 48 working electrodes (20A to 20D) and a portion of the counter electrode 28.

The entire conductive pattern is coated with a 15 μm to 20 μm thick passivation layer 22 (FIG. 2b) made of structurable, optically curable paint (2-component solder mask, Elpemer GL 2467 SM-DG, from the Peters company). In the passivation layer, through high-energy pulses of an excimer laser, clearances 24, 24A to 24D are introduced into the paint that serve to receive the biomolecules 26. In a passivation layer having a thickness of 15 μm to 20 μm, to remove the paint and to briefly melt the surface, about 130 20-ns pulses of an excimer laser (Lambda Physik) having a fluence of 600-1200 mJ/cm² are needed. The melting of the surface leads to the closure of surface pores of the gold layer, to a reduction of the surface roughness and, through ablation of a few gold layers, to the removal of surface impurities. The laser irradiation of the substrate can occur directly or through a lens system or mask, and facilitates clearances of any size and geometry.

FIG. 2b shows a section through a conductor path substrate having 3 identical spots. Each of the conductor paths 20 consists of a copper core 14 that is continuously coated by a nickel barrier layer 16 and a gold layer 18. In the exemplary embodiment, the copper core has a thickness of about 28 μm. It constitutes an economical and highly conductive main component of the conductor paths. To facilitate very precise measurements for electrochemical detection in an aqueous medium, the base copper core is coated with a 6 μm thick, continuous nickel layer as a diffusion barrier. On this nickel layer is applied 2 μm thick gold layer.

The conductor paths of the exemplary embodiment are about 150 μm wide and are disposed on the support plate with spacing of about 200 μm (center to center). The working electrodes, the counter electrode and a reference electrode that is provided if appropriate are each joined with connecting contact surfaces, which are not shown, of the electrical substrate for contact.

In the exemplary embodiments, the conductor paths have circular clearances with diameters of 10 μm, 30 μm, 100 μm and rectangular clearances measuring 100 μm×700 μm (cf. 24A to 24D in FIG. 2a). These clearances thus exhibit surface areas of 78.5 μm², 706.5 μm², 7,850 μ² or 70,000 μ², such that the active surface area of the electrodes is varied by about a factor of 1,000.

### EXAMPLE 2

Functionalizing the Substrate Spots with Nucleic Acid Oligomers

The free substrate sites of different sizes described in example 1 are functionalized with the nucleic acid oligomers, for example via a spotting method.

The synthesis of the oligonucleotides occurs in an automatic oligonucleotide synthesizer (Expedite 8909; ABI 384 DNA/RNA synthesizer) according to the synthesis protocols recommended by the manufacturer for a 1.0 μmol synthesis. In the syntheses with the 1-O-dimethoxytrityl-propyl-disulfide-CPG support (Glen Research 20-2933), the oxidation steps are carried out with a 0.02 mol/l iodine solution to avoid oxidative cleavage of the disulfide bridge. Modifications at the 5′-position of the oligonucleotides occur with a coupling step extended to 5 min. The amino modifier C2 dT (Glen Research 10-1037) is built into the sequences according to the respective standard protocol. The coupling efficiencies are determined online during the synthesis, photometrically or conductometrically, via the DMT cation concentration.

The oligonucleotides are deprotected with concentrated ammonia (30%) at 37° C. for 16 h. The purification of the oligonucleotides occurs by means of RP-HPL chromatography according to standard protocols (mobile phase: 0.1 mol/l triethylammonium acetate buffer, acetonitrile), and characterization by means of MALDI-TOF MS. The amine-modified oligonucleotides are coupled to the activated redox labels (e.g. osmium complexes) in accordance with the conditions known to the person skilled in the art. The coupling can occur either prior to or after the attachment of the oligonucleotides to the surface.

The substrates in example 1 are applied, for example, with doubly modified 20-bp single-strand oligonucleotide having the sequence 5′-AGC GGA TAA CAC AGT CAC CT-3′(modification one: the phosphate group of the 3′-end is esterified with (HO—(CH₂)₂—S)₂ to form P—O—(CH₂)₂—S—S—(CH₂)₂—OH. Modification two: the osmium complex [Os(bipy)₂ Cl imidazoleacrylic acid] is built into the amino-modified 5′-end according to the corresponding standard protocol) as a 5×10⁻⁵ molar solution in buffer (phosphate buffer, 0.5 molar in water, pH 7 with 0.05 vol % SDS) with the aid of a spotter (Cartesian) and incubated for 2-24 h.

During this reaction time, the disulfide spacer P—O—(CH₂)₂—S—S—(CH₂)₂—OH of the oligonucleotide is homolytically cleaved. Here, the spacer forms a covalent Au—S bond with Au atoms of the surface, thus causing a 1:1 coadsorption of the ss-oligonucleotide and the cleaved 2-hydroxy-mercaptoethanol. Instead of the single-strand oligonucleotide, the single-strand can also be hybridized with its complementary strand.

For loading with the spotter from Cartesian Technologies (MicroSys PA), split pins (Arraylt Chipmaker pins from TeleChem) are used that have a loading volume of 0.2 to 0.6 μl and dispense volumes of about 1 nl per wetting process. The contact surface of these pins has a diameter of about 130 μm and is thus considerably larger than the substrate regions exposed by laser ablation. The positioning of the pins above the substrate occurs with a precision of 10 μm at a humidity of about 70-80%. The droplet is dispensed upon contact of the tip with the passivation layer and no direct contact occurs with the substrate (“pseudo-contact printing”).

### EXAMPLE 3

Varying the Surface Loading through Coadsorbates

It is possible to reduce the loading density of a spot with nucleic acid oligomers in a controlled manner through coadsorption with thiols, and thus to increase the relative proportion of binding events while the target concentration and electrode size remain constant.

There are two methods to choose from for the coadsorption of thiols. In one method, the incubation solution consists of the nucleic acid oligomers (analogous to example 2) with additionally between approximately 10⁻⁵ to 10⁻¹ molar propanethiol. This simultaneously present, free propanethiol is coadsorbed by forming an Au—S bond and thus takes up space on the sensor surface. In an alternative method, the propanethiol (10⁻⁵ to 10⁻¹ molar in 500 mmol/l phosphate buffer) is applied in a second incubation step (30 min to 12 h) following the functionalization of the sensor surface with nucleic acid oligomers.

Through the use of propanethiol as the coadsorbate, the surface loading density of the nucleic acid oligomers in both variants can be reduced by up to a factor of 10.

### EXAMPLE 4

Varying the Surface Loading through Loading Parameters

It is also possible to adjust the surface loading density of the sensor spots by varying selected loading parameters when functionalizing with nucleic acid oligomers.

If, for example, the concentration of the nucleic acid oligomers in the incubation solution (500 mmol/l buffer) is increased from 1 μmol/l to 30 μmol/l, the loading density will increase by a factor of 5. A similar increase in the surface loading is achieved when the concentration of the incubation buffer is increased from 10 mmol/l to 500 mmol/l for a probe concentration of 30 μmol/l.

### EXAMPLE 5

Hybridization with Complementary Nucleic Acid Oligomers

A substrate having 48 working electrodes is produced having active regions of various sizes, as described in example 1. In groups of 12 electrodes each, circular holes having a diameter of 10 μm (spot group 1), 30 μm (spot group 2) and 100 μm (spot group 3) and a rectangular contour of 100 μm×700 μm (spot group 4) are burned with the excimer laser. The individual spot groups thus exhibit surface areas of 78.5 μm², 706.5 μm², 7,850 μm² and 70,000 μm², such that the surface area is varied by a factor of about 1,000.

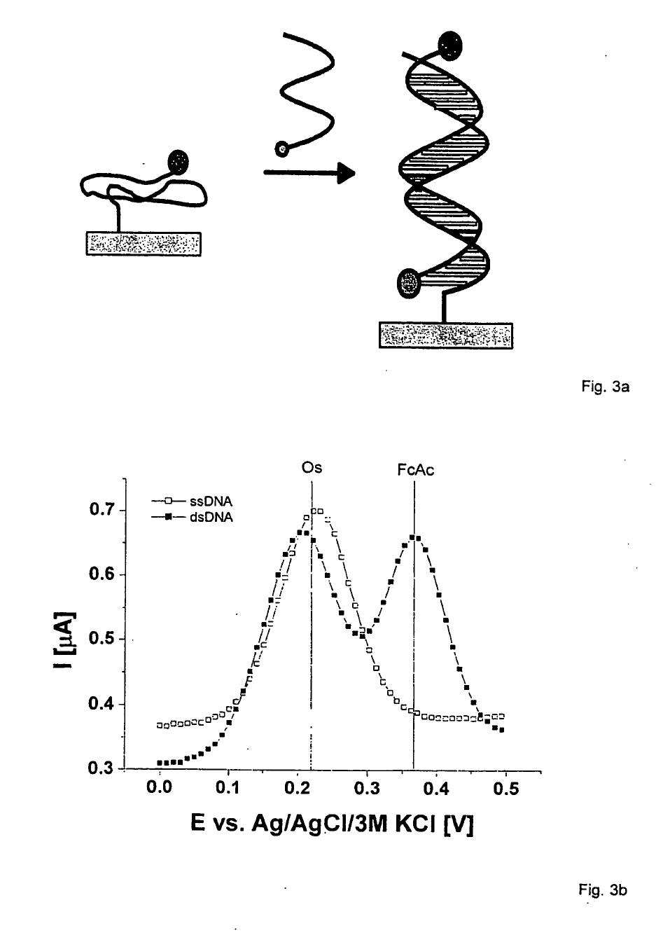

The working electrodes of a spot group are each functionalized with doubly modified nucleic acid oligomers (probes) of a certain sequence (S1 to S4), analogous to example 2. The working electrodes exhibit nearly identical surface loading densities. FIG. 3 shows, by way of example, an ACV measurement (U_{ac}=10 mV, f=5 Hz) of a working electrode (□ symbols in FIG. 3) that is functionalized with osmium-modified oligomers. It is possible to calculate the surface loading density with nucleic acid oligomers from the redox current at the potential of the osmium complex. In the present case, a value of 5×10⁻¹² mol/cm² results.

In a reloading step, after functionalization and before hybridization with complementary, ferrocene-modified nucleic acid oligomers, the working electrodes are brought into contact with a 1 mmol/l solution of propanethiol for 30 minutes. Here, the spaces between the nucleic acid oligomers are hydrophobized. As a result, the redox potential of the ferrocene shifts to more positive values, thus achieving a better separation from the osmium potential.

However, analogously to example 2, the four different target nucleic acid oligomers are synthesized without a thiol modification at the 3′-end. The target nucleic acid oligomers exhibit a sequence that is complementary to one probe nucleic acid oligomer in each case (T1 to T4). For the modification with the redox label ferrocene, the amino-modified nucleic acid oligomers at the 5′-end are coupled with ferrocene acetic acid (FcAc) in accordance with the respective standard protocol.

The four different ferrocene-modified nucleic acid oligomers are added to the target solution in different concentrations (T1=0.1 μmol/l, T2=1 μmol/l, T3=10 μmol/l, T4=100 μmol/l in 500 mmol/l phosphate buffer, pH 7, with 1 mol/l NaCl and 0.05 vol % SDS) and applied to all sensor spots. After a certain incubation time under hybridization conditions, the substrate is rinsed and an electrochemical ACV measurement (U_{ac}=10 mV, f=5 Hz) is once again conducted (▪ symbols in FIG. 3). The measurement data show a second redox peak, the ratio of the peak currents of the osmium label and the ferrocene label corresponding to the hybridization efficiency of the experiment.

The measurement data of the hybridization in FIG. 3 show an electrode close to saturation with a hybridization efficiency of more than 90%. The working electrodes having the sizes adjusted to the concentrations of the respective targets, on the other hand, all show the same hybridization efficiencies of about 30-40%.

### EXAMPLE 6

Diagnostic Chip

In medical diagnostics, it is desirable to capture multiple diagnostically relevant parameters simultaneously in one examination. An important example from the field of routine examinations is a vaginal smear, which is analyzed for HPV, E. coli and lactobacilli. For such a smear, with the aid of standardized swabs, a sample is taken that is then treated with standardized methods to obtain the RNA of the existing bacteria and the double-strand DNA of the viruses. In an examination, germ or particle counts up to a certain limit are classified as harmless: for HPV, this is 100 particles, for E. coli, 100 germs, and for lactobacilli, 10,000 germs of all relevant lactobacilli. Since, in bacteria cells, the characteristic RNAs each occur about 10⁴ times, a parallelized chip test must be able to capture very different concentrations to obtain all parameters simultaneously in one examination. A sensor chip according to the present invention for the above application has three different spot sizes that are functionalized with probe polynucleotides that are specific for the respective disease targets. To detect the HPV DNA in the range from 10² to 10⁴ molecules in the test substance, spots having a surface area of 1 μm² are used, while for the E. coli RNA in the range from 10⁶ to 10⁸ molecules (corresponding to 10² to 10⁴ germs), surface areas of 10⁴ μm², and for the lactobacilli RNA in the range from 10⁸ to 10¹⁰ molecules (corresponding to 10⁴ to 10⁶ germs), surface areas of 10⁶ μm² are used. It is ensured, through the selection of the electrode sizes, that the sensor permits quantitative measurements for the respective range starting at the critical target concentrations of the different pathogens, and thus that a parallel diagnosis of all diseases can be reached.

1. A substrate for use as a ligate carrier in a method for detecting ligate-ligand association events, having test sites (24) disposed on the substrate and having ligates (26) bound to the surface of the test sites (24), at least two types of test sites (24) being provided, the different types of test sites each being loaded with different types of ligates (26), the different types of ligates (26) detecting the respective complementary types of ligands, the ligands being present in an analyte solution in different concentration ranges in each case, and the test sites (24) exhibiting a characteristic loading parameter that permits detection of the ligands in their respective concentration range.

2. The substrate according to claim 1, wherein the characteristic loading parameter is the surface area of the test sites.

3. The substrate according to claim 2, wherein the surface area of the test sites (24) differs by at least a factor of 10.

4. The substrate according to claim 2, wherein the surface area of the test sites (24) differs by at least a factor of 100.

5. The substrate according to claim 2, wherein the surface area of the test sites (24) differs by at least a factor of 1,000.

6. The substrate according to claim 2, wherein the surface area of the test sites (24) differs by at least a factor of 10,000.

7. The substrate according to claim 2, wherein the surface area of the test sites (24) measures between 1 μm² and 10 mm².

8. The substrate according to claim 7, wherein the surface area of the test sites (24) measures between 10 μm² and 100,000 μm².

9. The substrate according to one of the preceding claims, wherein the characteristic loading parameter is the loading density of the test sites with ligates.

10. The substrate according to claim 9, wherein the loading density of the test sites (24) with ligates differs by at least a factor of 10.

11. The substrate according to claim 9, wherein the loading density of the test sites (24) with ligates differs by at least a factor of 100.

12. The substrate according to claim 9, wherein the loading density of the test sites (24) with ligates differs by at least a factor of 500.

13. The substrate according to claim 1, wherein the respective mean values of the concentration ranges in which the different types of ligands are present differ by at least a factor of 10.

14. The substrate according to claim 13, wherein the respective mean values of the concentration ranges in which the different types of ligands are present differ by at least a factor of 100.

15. The substrate according to claim 13, wherein the respective mean values of the concentration ranges in which the different types of ligands are present differ by at least a factor of 1,000.

16. The substrate according to claim 13, wherein the respective mean values of the concentration ranges in which the different types of ligands are present differ by at least a factor of 10,000.

17. The substrate according to claim 1, wherein cofactors or coenzymes are used as ligands, and proteins or enzymes are used as ligates.

18. The substrate according to claim 1, wherein antibodies are used as ligands, and antigens are used as ligates.

19. The substrate according to claim wherein antigens are used as ligands, and antibodies are used as ligates.

20. The substrate according to claim 1, wherein receptors are used as ligands, and hormones are used as ligates.

21. The substrate according to claim 1, wherein hormones are used as ligands, and receptors are used as ligates.

22. The substrate according to claim 1, wherein nucleic acid oligomers are used as ligands, and nucleic acid oligomers that are complementary thereto are used as ligates.

23. The substrate according to claim 1, wherein the substrate is loaded with a passivation layer that exhibits clearances at the test sites (24).

24. A method for detecting a ligate-ligand association event comprising:
providing a substrate having test sites (24) disposed on the substrate and having ligates (26) bound to the surface of the test sites (24), at least two types of test sites (24) being provided, the different types of test sites each being loaded with different types of ligates (26), the different types of ligates (26) detecting the respective complementary types of ligands, the ligands being present in an analyte solution in different concentration ranges in each case, and the test sites (24) exhibiting a characteristic loading parameter that permits detection of the ligands in their respective concentration range; and
using the substrate in a method for detecting ligate-ligand association events.

25. The use according to claim 24, wherein the method for detecting ligate-ligand association events is an electrochemical detection method selected from the group selected from the group consisting of chronoamperometry (CA), chronocoulometry (CC), linear sweep voltammetry (LSV), cyclic voltammetry (CSV), alternating current voltammetry (ACV), voltammetry techniques with different pulse shapes, differential pulse voltammetry (DPV) or normal pulse voltammetry (NPV), AC or DC impedance spectroscopy, chronopotentiometry and cyclic chronopotentiometry.

26. The method according to claim 24, wherein the method for detecting ligate-ligand association events is a fluorescent spectroscopic detection method.
